(19) United States
(12) Patent Application Publication
(10) Pub. No.: US 2005/0182036 A1
(43) Pub. Date: Aug. 18, 2005

(54) Medicinal composition containing an HMG-CoA reductase inhibitor

(75) Inventors: Tatsuhito Kondo, Tokyo (JP); Ikuo Takagi, Matsudo-shi (JP); Masato Nakayama, Kitakatsushika-gun (JP); Yasuhiro Torizumi, Ryugasaki-shi (JP)

Correspondence Address: FRISHAUF, HOLTZ, GOODMAN & CHICK, PC, 220 5TH AVE FL 16, NEW YORK, NY, 10001-7708, US

(73) Assignee: SANKYO COMPANY, LIMITED, Tokyo (JP)

(21) Appl. No.: 11/045,400

(22) Filed: Jan. 27, 2005

Related U.S. Application Data
(63) Continuation-in-part of application No. PCT/JP03/09835, filed on Aug. 1, 2003.

(30) Foreign Application Priority Data
Aug. 2, 2002 (JP) 2002-225979
Sep. 6, 2002 (JP) 2002-260719

(51) Int. Cl. A61K 31/56; A61K 31/51; A61K 31/401; A61K 31/366; A61K 31/225

(52) U.S. Cl. 514/171; 514/423; 514/460; 514/548; 514/276

(57) ABSTRACT

A pharmaceutical composition for promoting the synthesis of vascular endothelial nitrogen oxide and/or maintaining or elevating the concentration of vascular endothelial nitrogen oxide in the blood, or a pharmaceutical composition for improving blood lipids. The pharmaceutical composition contains an HMG-CoA reductase inhibitor; and gamma-oryzanol, and/or thiamines. For treatment, the components of the composition can be administered together, as a composition, or separately.

This is a Continuation-in-Part Application of International Application No. PCT/JP2003/009835 filed Aug. 1, 2003, incorporated by reference herein in its entirety.

### BACKGROUND OF THE INVENTION

The present invention relates to a medicinal composition comprising an HMG-CoA reductase inhibitor and γ-oryzanol and/or thiamine derivatives, (particularly a medicinal composition for promoting the production of vascular endothelial nitric oxide and/or maintaining or elevating the concentration of vascular endothelial nitrogen oxide in the blood, or a medicinal composition for mitigating blood lipid).

Since old times it has been said that a man is as old as his arteries. Recently, it has been noticed that there is a significant relationship between decreases in vascular endothelial nitric oxide (NO) production due to aging and various diseases frequently observed in elderly individuals. This suggests that vascular aging may be related to decreases of endothelial nitric oxide synthase (eNOS) activity.

It has been reported that functional impairment of the vascular endothelium is strongly related to pathogenesis and progression of atherosclerosis, which is largely caused by decreases in NO production generated by eNOS. Nitric oxide derived from the vascular wall exhibits several anti-atherosclerotic effects resulting from concomitant vasodilatation, inhibition of various factors such as platelet coagulation, adhesion of blood neutrophils to endothelial cells, migration and proliferation of vascular smooth muscle cells, as well as suppression of oxidation of LDL, and the like (see for example; The Journal of Japanese College of Angiology, Vol. 38 No. 4, 1998 p.215-216).

Since atherosclerosis has been demonstrated to be exacerbated by blocking NO synthesis in animal experiments, some direct contribution from NO production seems likely in the pathogenesis as well as progression of atherosclerosis (see for example, Vascular Biology & Medicine, Vol. 12, No. 2, 2001, p. 189).

Since NO produced in endothelial cells exerts various vascular protective actions in humans, these effects may be exploited in therapeutic strategies designed to maintain and/or improve vascular endothelial function. Examples of known agents that promote eNOS activity include statins, L-arginine, ACE inhibitors, angiotensin II type 1 receptor antagonists, hormones, and some calcium antagonists. In addition, antioxidants such as vitamin C, vitamin E, probucol, and the like have been reported to be beneficial because they indirectly promote NO action by preventing inactivation of NO (see for example, Japanese pharmacology & Therapeutics, Vol. 29, No. 10, 2001, p. 716).

Furthermore, it has been shown that vitamin C increases eNOS activity (see for example, Vitamin Vol. 75, No.2, 2001, p. 511).

In addition, ginseng, Astragalus root, and scutellariae radix of galenical composition in Chinese herbal medicine have been found to stimulate NO production in blood vessels (see for example, Journal of Traditional Medicines, Vol. 11, 1994, p. 102).

Moreover, nitric oxide synthase (NOS) is localized not only in the vascular vessels but also in many tissues other than blood vessels, and plays important roles in the regulation of the cardiovascular system. There are many diseases in which NO production is lowered and as such diseases the following diseases are well known: cardiovascular diseases such as hypertension, hypercholesterolemia, atherosclerosis, ischemic heart disease, cardiac failure, thrombosis, and the like; respiratory diseases such as asthma, chronic obstructive pulmonary disease, pulmonary hypertension, ARDS (Adult respiratory distress syndrome), and the like; diseases of digestive organs such as hepatopathy, cirrhosis, gastrointestinal mucous disorders, hypertrophic pyloric stenosis, pancreatitis, and the like; cerebrovascular disorders such as cerebral ischemia, cerebral infarction, cerebral circulation disorders, senile dementia, and the like; disorders of the kidney and urinary tract such as renal dysfunction, impotency, and the like; disorders of obstetrics & gynaecology such as gestational toxicosis, and the like; infectious and immune diseases, diabetes mellitus, burn injury, and the like. As other factors, it has also been known that NO production is lowered by certain drugs (see for example, Japanese Patent Publication (Kokai) Number Hei 10-338637).

Statins are remedies that reduce blood cholesterol levels by inhibiting specifically and competitively HMG-CoA reductase in vivo. In addition to these effects, statins have also been known to promote eNOS activity. However, gamma-oryzanol and thiamines have not been reported as having promoting effects on eNOS activity. Furthermore, there are no data on synergistic effects of a combination treatment using a statin and a thiamine derivative on the production and/or on maintaining or increasing blood concentration of vascular endothelial nitric oxide.

Moreover, it is not known whether a combination of a statin and gamma-oryzanol and/or a thiamine derivative lowers blood lipids synergistically.

### BRIEF SUMMARY OF THE INVENTION

In light of this background, the present inventors have eagerly studied pharmacological actions of a combination of an HMG-CoA reductase inhibitor and gamma-oryzanol and/or a thiamine derivative and found that the production of vascular endothelial nitric oxide was promoted and the blood concentration of vascular endothelial nitric oxide was highly maintained or increased, and blood lipids were mitigated, and completed the present invention.

The present invention relates to
(1) a medicinal composition comprising an HMG-CoA reductase inhibitor and gamma-oryzanol and/or a thiamine derivative as active ingredients.

Of the said description, the medicinal compositions of the present invention include
(2) a medicinal composition according to (1), comprising one or more HMG-CoA reductase inhibitors selected from the group consisting of pravastatin, lovastatin, simvastatin, fluvastatin, rivastatin, atorvastatin, pitavastatin, and rosuvastatin as active ingredients,
(3) a medicinal composition according to (1), comprising one or more HMG-CoA reductase inhibitors selected from the group consisting of simvastatin and atorvastatin as active ingredients,
(4) a medicinal composition according to any one selected from (1) to (3) described above, comprising one or more thiamine derivatives selected from the group consisting of thiamine, dicethiamine, octotiamine, cycotiamine, bisibuthiamine, bisbenthiamine, fursultiamine, prosultiamine, benfotiamine, and pharmacologically acceptable salts thereof,
(5) a medicinal composition according to any one selected from (1) to (3) described above, in which the thiamine derivative is benfotiamine,
(6) a medicinal composition according to any one selected from (1) to (5) for promoting the production of vascular endothelial nitric oxide and/or maintaining or increasing blood concentration of vascular endothelial nitric oxide,
(7) a medicinal composition according to any one selected from (1) to (5) for mitigating blood lipids,
(8) a medicinal composition as stated in (7), in which the HMG-CoA reductase inhibitor is simvastatin,
(9) a medicinal composition according to any one selected from (1) to (5) for the prevention or treatment of diseases caused by decreases in vascular endothelial nitric oxide synthase activity and/or lowered blood concentration of vascular endothelial nitric oxide,
(10) a medicinal composition according to any one selected from (1) to (5) to prevent or to treat diseases that decrease vascular endothelial nitric oxide synthase activity and/or blood concentration of vascular endothelial nitric oxide,
(11) a medicinal composition according to any one selected from (1) to (5) for the prevention or treatment of decreases in NO production caused by cardiovascular diseases, cerebrovascular diseases, disorders of the kidney and urinary tract, diabetes mellitus, or certain drugs.
(12) a medicinal composition according to any one selected from (1) to (5) for the prevention or treatment of diseases caused by high blood lipid levels,
(13) a medicinal composition according to (12), in which an HMG-CoA reductase inhibitor and gamma-oryzanol are contained as essential active ingredients, and
(14) a medicinal composition according to any one selected from (1) to (5) for the prevention or treatment of hypercholesterolemia or atherosclerosis.

In addition, the present invention provides
(15) a combination therapy of an HMG-CoA reductase inhibitor and gamma-oryzanol and/or a thiamine derivative to promote the production of vascular endothelial nitric oxide and/or to maintain or increase vascular nitric oxide concentration in the blood by administration of an HMG-CoA reductase inhibitor and gamma-oryzanol and/or a thiamine derivative simultaneously or separately at certain time intervals,
(16) a combination therapy of an HMG-CoA reductase inhibitor and gamma-oryzanol and/or a thiamine derivative to mitigate blood lipids by administration of an HMG-CoA reductase inhibitor and gamma-oryzanol and/or a thiamine derivative simultaneously or separately at certain time intervals,
(17) a combination therapy of an HMG-CoA reductase inhibitor and gamma-oryzanol and/or a thiamine derivative to promote the production of vascular endothelial nitric oxide and/or to maintain or increase blood vascular nitric oxide concentration,
(18) a combination therapy of an HMG-CoA reductase inhibitor and gamma-oryzanol and/or a thiamine derivative to mitigate blood lipid levels.

Furthermore, the present invention provides
(19) a method to promote the production of vascular endothelial nitric oxide and/or to maintain or increase blood vascular nitric oxide concentration by administration of an HMG-CoA reductase inhibitor and gamma-oryzanol and/or a thiamine derivative simultaneously or separately at certain time intervals, and
(20) a method to mitigate blood lipid levels by administration of an HMG-CoA reductase inhibitor and gamma-oryzanol and/or a thiamine derivative simultaneously or separately at certain time intervals.

The preferable methods according to (19) are
(21) a method according to (19) to prevent or treat diseases caused by decreases in vascular endothelial nitric oxide synthase activity and/or lowered blood concentration of vascular endothelial nitric oxide,
(22) a method according to (19) to prevent or treat diseases that decrease vascular endothelial nitric oxide synthase activity and/or blood concentration of vascular endothelial nitric oxide, and
(23) a method according to (19) to prevent or treat decreases in vascular endothelial nitric oxide synthase activity and/or lowered blood concentration of vascular endothelial nitric oxide caused by cardiovascular diseases, cerebrovascular disorders, disorders of the kidney and urinary tract, diabetes mellitus, and certain drugs.

Of the said methods (20), preferable are
(24) a method as stated in (20) to prevent or treat diseases caused by high blood lipid levels, and
(25) a method as stated in (20) to prevent or treat hypercholesterolemia, or atherosclerosis.

Furthermore, the present invention provides
(26) the use of an HMG-CoA reductase inhibitor and gamma-oryzanol and/or a thiamine derivative in the manufacture of a medicinal composition comprising an HMG-CoA reductase inhibitor and gamma-oryzanol and/or a thiamine derivative for promoting activity of vascular endothelial nitric oxide synthase and/or maintaining or increasing blood concentration of vascular endothelial nitric oxide, and
(27) the use of an HMG-CoA reductase inhibitor and gamma-oryzanol and/or a thiamine derivative in the manufacture of a medicinal composition comprising an HMG-CoA reductase inhibitor and gamma-oryzanol and/or a thiamine derivative for mitigating blood lipids.

### DETAILED DESCRIPTION OF THE INVENTION

“HMG-CoA reductase inhibitor”, which is one component of the medicinal composition of the present invention, refers to agents that competitively and specifically inhibit 3-hydroxy-3-methyl-glutaryl-CoA (HMG-CoA) reductase, which is a rate limiting enzyme in the biosynthesis of cholesterol. Since such inhibitors suppress blood cholesterol levels, the inhibitors are used as therapeutic agents for patients with hypercholesterolemia. As such HMG-CoA reductase inhibitors, natural products derived from microorganisms and semi-synthesized compounds derived from the natural products described above, and totally synthesized chemical compounds are all included. For instance, such compounds are (+)-(3R, 5R)-3,5-dihydroxy-7[(1S,2S,6S,8S,8aR) -6-hydroxy-2-methyl-8-[(S) -2-methylbutyryloxy]-1,2,6,7,8,8a-hexahydro-1-naphtyl] heptanoic acid which is disclosed in Japanese Patent Publication (Kokai) Number Sho 57-2240 (U.S. Pat. No. 4,346,227) (hereinafter called pravastatin),

(+)-(1S,3R,7S,8S,8aR)-1,2,3,7,8,8a-hexahydro-3,7-dimethyl-8-[2-[ (2R,4R)-tetrahydro-4-hydroxy-6-oxo-2H-pyran-2-yl]ethyl]-1-naphth yl(S)-2-methylbutyrate which is disclosed in Japanese Patent Publication (Kokai) Number Sho 57-163374 (U.S. Pat. No. 4,231,938) (hereinafter called lovastatin),

(+)-(1S,3R,7S,8S,8aR)-1,2,3,7,8,8a-hexahydro-3,7-dimethyl-8-[2-[ (2R,4R)-tetrahydro-4-hydroxy-6-oxo-2H-pyran-2-yl]ethyl]-1-naphth yl 2,2-dimethylbutyrate which is disclosed in Japanese Patent Publication (Kokai) Number Sho 56-122375 (U.S. Pat. No. 4,444,784) (hereinafter called simvastatin),

(±)(3R*,5S*,6E)-7-[3-(4-fluorophenyl)-1-(1-methylethyl)-1H-indol -2-yl]-3,5-dihydroxy-6-heptenoic acid which is disclosed in Japanese Patent Publication (Kohyo) Number Sho 60-500015 (U.S. Pat. No. 4,739,073) (hereinafter called fluvastatin),

(3R,5S,6E)-7-(4-(4-fluorophenyl)-2,6-di(1-methylethyl)-5-methoxy methylpyridin-3-yl]-3,5-dihydroxy-6-heptenoic acid which is disclosed in Japanese Patent Publication (Kokai) Number Hei 1-216974 (U.S. Pat. No. 5006530) (hereinafter called rivastatin),

(3R,5S)-7-[2-(4-fluorophenyl)-5-(1-methylethyl)-3-phenyl-4-pheny laminocarbonyl-1H-pyrrol-1-yl]-3,5-dihydroxyheptanoic acid which is disclosed in Japanese Patent Publication (Kokai) Number Hei 3-58967 (U.S. Pat. No. 5,273,995) (hereinafter called atorvastatin), and

(E)-3,5-dihydroxy-7-[4′-(4″-fluorophenyl)-2′-cyclopropyl-quinol in-3′-yl]-6-heptenoic acid which is disclosed in Japanese Patent Publication (Kokai) Number Hei 1-279866 (U.S. Pat. No. 5,854,259 and U.S. Pat. No. 5,856,336) (hereinafter called pitavastatin), or

(+)-(3R,5S)-7-[4-(4-fluorophenyl)-6-isopropyl-2-(N-methyl-N-meth anesulfonylamino)pyridin-5-yl]-3,5-dihydroxy-6(E)-heptenoic acid which is disclosed in Japanese Patent Publication (Kokai) Number Hei 5-178841 (U.S. Pat. No. 5,260,440) (hereinafter called rosuvastatin). In addition, an HMG-CoA reductase inhibitor, which is one component of the medicinal composition of the present invention, refers to other HMG-CoA reductase inhibitors described in the disclosed patents described above.

Planar chemical structures of representative HMG-CoA reductase inhibitors are shown below:

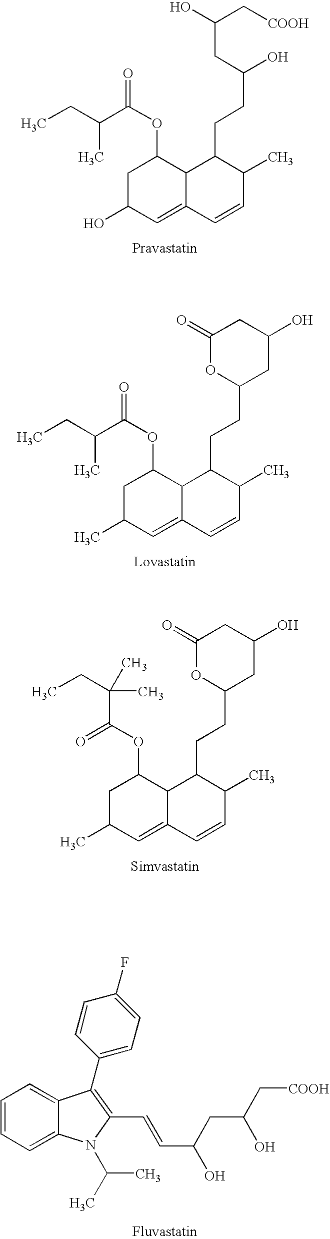

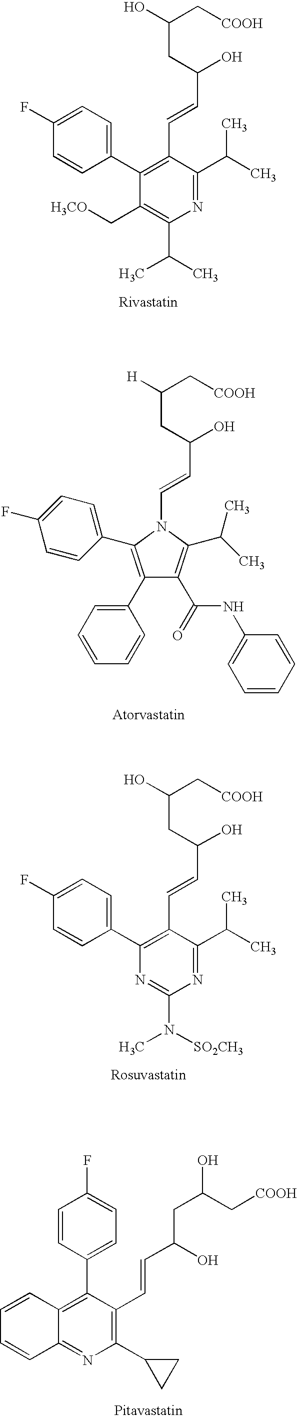

Of these HMG-CoA reductase inhibitors, simvastatin and atorvastatin are preferable, and simvastatin is more preferable.

“Gamma oryzanol” is mainly extracted from rice-bran oil and rice germ oil and is a sterol or ferulic acid compound esterified with triterpene alcohol or a mixture of these compounds.

“Thiamine derivatives” or “compounds” are thiamine, dicethiamine, octotiamine, cycotiamine, bisibuthiamine, bisbenthiamine, fursultiamine, prosultiamine, benfotiamine, or salts thereof, and preferably benfotiamine.

In the present invention, each active ingredient as described above may be present as pharmacologically acceptable salts thereof, and

in the case that the active ingredients present a basic functional group, such salts are, for example, a hydrohalide such as hydrofluoride, hydrochloride, hydrobromide, hydroiodide, or the like; an inorganic acid salt such as a nitrate, a perchlorate, a sulfate, a phosphate, or the like; a lower organic sulfonate such as methanesulfonate, trifluoromethanesulfonate, ethanesulfonate, or the like; an arylsulfonate such as benzenesulfonate p-toluenesulfonate, or the like; an amino acid salt such as ornithine salt, glutamate, or the like; carboxylic acid salt such as a fumarate, a succinate, a citrate, a tartrate, an oxalate, a maleate, or the like,

in the case that the active ingredients present an acidic functional group, such salts are, for example, an alkaline metal salt such as sodium salt, potassium salt, lithium salt, or the like; an alkaline earth metal salt such as calcium salt, magnesium salt, or the like; a metal salt such as an aluminium salt, an iron salt, a zinc salt, a copper salt, a nickel salt, a cobalt salt, or the like; an amine salt, for example, an inorganic amine salt such as ammonium salt, an organic amine salt such as t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzylphenethylamine salt, piperazine salt, tetramethylammonium salt, tris (hydroxymethyl) aminomethane salt, or the like. For instance, in the case of pravastatin, a preferable salt is pravastatin sodium, and for instance, in the case of atorvastatin, a preferable salt is atorvastatin calcium salt hydrate.

In the case that each active ingredient involved forms a hydrate or solvate, such hydrates or solvates are included in the medicinal compositions of the present invention.

In the present invention, “various diseases” means diseases caused by decreases in vascular endothelial nitric oxide synthase activity and/or lowered blood concentration of vascular endothelial nitric oxide, or diseases in which as the result of the diseases, vascular endothelial nitric oxide synthase activity is decreased and/or blood concentration of vascular endothelial nitric oxide is lowered. As such examples of these diseases, cardiovascular disorders such as hypertension, hypercholesterolemia, atherosclerosis, ischemic heart disease, cardiac failure, thrombosis, pulmonary hypertension, resteriosis, peripheral circulatory disorder or the like; cerebrovascular disorders such as pulmonary hypertension, cerebral ischemia, cerebral infarction, cerebral circulation disorders, senile dementia, or the like; disorders of the kidney and urinary tract such as renal disorder, impotence, or the like; diabetes mellitus, or certain drugs are included.

Since the early stages of the said diseases exhibit few subjective symptoms, it is difficult for patients themselves to become aware of having these diseases in the early stages. The subjective symptoms of the “various diseases” in the present invention are, for example, headache, dizziness, numbness or feeling of numbness, cold sensation of the four limbs, shoulder stiffness, skin atrophy and muscle atrophy, impotence, and the like. Therefore the said diseases may possibly be treated at their early stages by taking the medicinal composition of the present invention when such subjective symptoms first appear.

“Mitigating blood lipids” means reducing blood lipid levels to clinically significant values, that is, reducing blood triglyceride levels, reducing blood LDL levels, or reducing blood total cholesterol levels.

Preparation

HMG-CoA reductase inhibitors used as an active ingredient in the medicinal composition of the present invention, for example, pravastatin, lovastatin, simvastatin, fluvastatin, rivastatin, atorvastatin, pitavastatin, or rosuvastatin, can be easily prepared according to the methods described hereinafter in Japanese Patent Publication (Kokai) Number Sho 57-2240 (U.S. Pat. No. 4,346,227), Japanese Patent Publication (Kokai) Number Sho 57-16337 (U.S. Pat No. 4,231,938), Japanese Patent Publication (Kokai) Number Sho 56-122375 (U.S. Pat. No. 4,444,784), Japanese Patent Publication (Kohyo) Number Sho 60-500015 (U.S. Pat. No. 4,739,073), Japanese Patent Publication (Kokai) Number Hei 1-216974 (U.S. Pat. No. 5,006,530), Japanese Patent Publication (Kokai) Number Hei 3-58967 (U.S. Pat. No. 5,273,995), Japanese Patent Publication (Kokai) Number Hei 1-279866 (U.S. Pat. No. 5,854,259 and U.S. Pat. No. 5,856,336), or Japanese Patent Publication (Kokai) Number Hei 5-178841 (U.S. Pat. No. 5,260,440).

In addition, gamma-oryzanol contained in the medicinal compositions of the present invention as an active ingredient can be easily obtained as a commercially available product, or can be easily manufactured by previously known methods.

Furthermore, thiamine derivatives can be easily obtained, since their specifications are disclosed in “The Japanese Pharmacopoeia (JP) 14th Edition”.

The medicinal compositions of the present invention comprising an HMG-CoA reductase inhibitor and gamma-oryzanol and/or thiamine derivatives as active ingredients contain both an HMG-CoA reductase inhibitor and gamma-oryzanol and/or a thiamine derivative as essential active ingredients. Additive agents may be contained in the formulation as required. In addition, other active ingredients contained in the medicinal compositions are not particularly restricted provided that they have no adverse effects when they are co-administered with the HMG-CoA reductase inhibitor and gamma-oryzanol and/or thiamine derivative contained in the medicinal composition. The preferable medicinal composition is restricted to an HMG-CoA reductase inhibitor and gamma-oryzanol and/or a thiamine derivative alone as active ingredients, and it may contain additive agents for its formulation.

The concrete preparations of the medicinal composition of the present invention are, for example, tablets, granules (including powders), capsules, liquids and solutions (including syrups), and the like. These preparations are prepared by conventionally known methods disclosed in “The Japanese Pharmacopoeia (JP)” or the like using additive agents and bases that are suitable for each preparation, as necessary.

In each preparation, various conventionally used additive agents suitable for each preparation may also be used.

For example, in the case of tablets, diluents such as lactose, crystalline cellulose, or the like; stabilizers such as magnesium aluminometasilicate, magnesium oxide, or the like; coating agents such as hydroxypropylcellulose, or the like; and lubricants such as magnesium stearate, or the like may be used.

In the case of granules and capsules, diluents such as lactose, purified sucrose, or the like; stabilizers such as magnesium aluminometasilicate, magnesium oxide, or the like; adsorbents such as corn starch, or the like; and binders such as hydroxypropylcellulose, or the like may be used.

In each preparation described above, disintegrants such as crospovidone, or the like; surfactants such as polysorbate, or the like; adsorbents such as calcium silicate, or the like; colouring agents such as red ferric oxide, caramel, or the like; stabilizers such as sodium parahydroxybenzoate, or the like; pH modifiers; flavours or the like may be added as necessary.

In the present invention, “co-administration” means methods of administration of 2 or more active ingredients to humans simultaneously, or separate administration of 2 or more active ingredients described above at a certain time interval.

When the active ingredients of the present invention are administered, each active ingredient of the medicinal composition may be administered simultaneously or separately at a certain time interval.

“Administration simultaneously” described above includes administration of each active ingredient at a pharmacologically acceptable time interval in addition to administration of all active ingredients at the same time. There is no restriction provided that their pharmaceutical preparations are to be taken at roughly the same time. Nevertheless, it is favourable to take the active ingredients as a single pharmaceutical preparation.

“Separate administration of the active ingredients at a certain time interval” described above has no restriction provided that their available pharmaceutical preparations are to be taken independently at different times. For instance, it indicates that first one active ingredient is administered, and then after a defined time delay, the other active ingredient is administered.

Furthermore, in the case that the medicinal composition contains 3 or more active ingredients, “simultaneous administration or separate administration at certain time intervals” includes all cases wherein all active ingredients contained in the medicinal composition are taken simultaneously, each active ingredient is taken separately at certain time delays, 2 or more active ingredients contained in the medicinal composition are simultaneously taken and the rest of them are separately taken at certain time intervals, or 2 or more active ingredients in the medicinal composition are simultaneously taken and the rest of them are simultaneously taken after a certain time delay.

Since the medicinal composition of the present invention comprising an HMG-CoA reductase inhibitor and gamma-oryzanol and/or a thiamine derivative exerts remarkable promoting effects on vascular endothelium nitric oxide synthase activity, maintaining effects or increasing effects on blood concentration of vascular endothelium nitric oxide, and mitigating effects on blood lipids, the medicinal compositions of the present invention are useful as pharmaceutical agents to prevent or to treat diseases caused by decreased activity of vascular endothelium nitric oxide synthase, and/or lowered blood concentration of vascular endothelium nitric oxide, diseases wherein decreases in vascular endothelial NOS activity, and/or decreases in concentration of vascular endothelial NO are elicited, or diseases caused by high blood lipid levels. Thus the medicinal compositions of the present invention are useful as preventive or therapeutic agents against diseases with symptoms of decreased activity of vascular endothelium nitric oxide synthase, and/or lowered blood concentration of vascular endothelium nitric oxide, which are caused by diseases, for example, cardiovascular disorders such as hypertension, hypercholesterolemia, atherosclerosis, ischemic heart disease, cardiac failure, thrombosis, pulmonary hypertension, restenosis, peripheral circulatory disorder, and the like; cerebrovascular disorders such as pulmonary hypertension, cerebral ischemia, cerebral infarction, cerebral circulation disorders, senile dementia, and the like; disorders of the kidney and urinary tract such as renal disorder, impotency, and the like; diabetes mellitus, or certain drugs.

In the present invention, the dosage of HMG-CoA reductase inhibitors varies depending on the types of HMG-CoA reductase inhibitors used, the formulations, and the like. It is usual to administer 0.015-3.5 mg/kg/day and preferably 0.08-2.7 mg/kg/day to a mammal. For example, it is usual to administer to an adult human, 1 mg-200 mg per day and preferably 5 mg-160 mg per day.

In the present invention, the dosage of gamma-oryzanol is usually 0.15-17 mg/kg/day and preferably 1.5-10 mg/kg/day to a mammal. For example, it is usual to administer to an adult human 10 mg-1,000 mg per day and preferably 100 mg-600 mg per day.

In the present invention, the dosage of thiamine derivatives varies depending on the types of thiamines used and their formulations. It is usual to administer 0.008-8.5 mg/kg/day and preferably 0.08-3.5 mg/kg/day to a mammal. For example, it is usual to administer to an adult human, 0.5 mg-500 mg per day and preferably 5 mg-200 mg per day.

In the case that the dosage form of the medicinal composition of the present invention is a solid dosage form, the weight percentages of the HMG-CoA reductase inhibitor contained in the medicinal composition is usually 0.005-3%, and preferably 0.03-2%, in the case of simvastatin, the weight percentage is usually 0.005-3%, and preferably 0.03-2%, and in the case of atorvastatin, the weight percentage is usually 0.01-5%, and preferably 0.05-3%.

In the case that gamma-oryzanol is present, the weight percentage is usually 0.5-90%, and preferably 3-60%,

In the case that a thiamine derivative is present, the weight percentage is usually 0.2-40%, and preferably 1-30%.

In the case that the dosage form of the medicinal composition of the present invention is a liquid or solution, the content of the HMG-CoA reductase inhibitor contained in the medicinal composition is usually 0.005-5 mg/mL, and preferably 0.03-3 mg/mL. For instance in the case of simvastatin, the content is usually 0.005 - 5 mg/mL, and preferably 0.03-3 mg/mL, while in the case of atorvastatin, the content is usually 0.01-10 mg/mL, and preferably 0.05-5 mg/mL.

In the case that gamma-oryzanol is present, the content is usually 2-200 mg/mL, and preferably 10-100 mg/mL.

In the case that a thiamine derivative is contained, the content is usually 1-100 mg/mL, and preferably 5-50 mg/mL.

### EXAMPLES

The present invention will further be exemplified in more detail by the Examples, and the like. However the scope of the present invention is not limited by these Examples.

### Example 1

### Tablets

(1) Compositions

| | 6 Tablets | 6 Tablets | |
|---|---|---|---|
| | (mg) | (mg) | 6 Tablets (mg) |
| Atorvastatin calcium | 20 | — | — |
| Simvastatin | — | 10 | 10 |
| Gamma-oryzanol | — | 300 | — |
| Benfotiamine | 100 | — | 100 |
| Magnesium oxide | 400 | 400 | 400 |
| Magnesium | 140 | 140 | 140 |
| aluminometasilicate | | | |
| Crystalline cellulose | 120 | 120 | 120 |
| Corn starch | 140 | 140 | 140 |
| Hydroxypropylcellulose | 60 | 60 | 60 |
| Croscarmellose sodium | 15 | 15 | 15 |
| Magnesium stearate | 25 | 25 | 25 |
| Glycerin triacetate | 6 | 6 | 6 |
| Lactose | A suitable | A suitable | A suitable |
| | amount | amount | amount |
| Total | 1,200 | 1,200 | 1,200 |

(2) Manufacturing Methods

Each active ingredient described above is weighed and the tablets are manufactured according to methods described in General Rules for Preparation (tablets) of the Japanese Pharmacopoeia.

### Example 2

### Granules

(1) Compositions

| | 3 Packages | 3 Packages | 3 Packages |
|---|---|---|---|
| | (mg) | (mg) | (mg) |
| Atorvastatin calcium | 20 | — | — |
| Simvastatin | — | 10 | 10 |
| Gamma-oryzanol | — | 300 | — |
| Benfotiamine | 100 | — | 100 |
| Magnesium oxide | 400 | 400 | 400 |
| Magnesium | 140 | 140 | 140 |
| aluminometasilicate | | | |
| Purified sucrose | 1400 | 1400 | 1400 |
| Extracted products from | 15 | 15 | 15 |
| stevia | | | |
| Corn starch | 1200 | 1000 | 1100 |
| Polysorbate 80 | 80 | 80 | 80 |
| Magnesium stearate | 25 | 25 | 25 |
| Lactose | A suitable | A suitable | A suitable |
| | amount | amount | amount |
| Total | 4,300 | 4,300 | 4,300 |

(2) Manufacturing Methods

Each active ingredient described above is weighed and the granules are manufactured according to methods described in General Rules for Preparation (granules) of the Japanese Pharmacopoeia.

### Example 3

### Capsules

(1) Compositions

| | 6 Capsules | 6 Capsules | 6 Capsules |
|---|---|---|---|
| | (mg) | (mg) | (mg) |
| Atorvastatin calcium | 20 | — | — |
| Simvastatin | — | 10 | 10 |
| Gamma-oryzanol | — | 300 | — |
| Benfotiamine | 100 | — | 100 |
| Magnesium oxide | 400 | 400 | 400 |
| Corn starch | 600 | 400 | 500 |
| Polysorbate 80 | 50 | 50 | 50 |
| Magnesium stearate | 25 | 25 | 25 |
| Lactose | A suitable | A suitable | A suitable |
| | amount | amount | amount |
| Capsule | 480 | 480 | 480 |
| Total | 2,300 | 2,300 | 2,300 |

(2) Manufacturing Methods

Each active ingredient described above is weighed and granules are manufactured according to methods described in General Rules for Preparation (granules) of the Japanese Pharmacopoeia. The capsules are manufactured by filling the granules in hard capsules.

### Example 4

### Syrups

(1) Compositions

| | 60 mL (mg) | 60 mL (mg) | 60 mL (mg) |
|---|---|---|---|
| Atorvastatin calcium | 20 | — | — |
| Simvastatin | — | 10 | 10 |
| Gamma-oryzanol | — | 300 | — |
| Benfotiamine | 100 | — | 100 |
| Sodium benzoate | 240 | 240 | 240 |
| Citric acid | 60 | 60 | 60 |
| Sucrose | 1,500 | 1,500 | 1,500 |
| Conc. Glycerin | 1,800 | 1,800 | 1,800 |
| Polyvinylalcohol | 120 | 120 | 120 |
| Ethanol (95%) | 500 | 9,000 | 4,500 |
| Hydrochloric acid | A suitable | A suitable | A suitable |
| | amount | amount | amount |
| Sodium hydroxide | A suitable | A suitable | A suitable |
| | amount | amount | amount |
| Purified water | A suitable | A suitable | A suitable |
| | amount | amount | amount |

(2) Manufacturing Methods

Each active ingredient described above is weighed and the syrups are manufactured according to methods described in General Rules for Preparation (syrups) of the Japanese Pharmacopoeia. The syrups are kept in brown glass bottles.

### Test Examples

Evaluation Test of Blood Nitric Oxides and Blood Lipid Level

### Test Example 1

(1) Test Substance

Simvastatin and atorvastatin synthesized at Chemtech Labo., Inc. were used. Gamma-oryzanol was purchased from Riken Vitamin Co., Ltd. and used. Benfotiamine manufactured at Sankyo Co., Ltd. was used.

(2) Animals

Male Beagle dogs of 5 months of age were purchased from Covance Research Products Inc. as the test animals and were used after accommodation breeding for approximately 5 months.

(3) Dosage Form, Preparation of the Formulation, and Storage

The required amount of the test substance calculated based on the body weight of the test animals was weighed and filled in gelatin capsules (TORPAC Inc., 1/2 oz). After filling up, the capsules were placed in a box divided up for every animal and stored under freezing until use.

(4) Route of Administration and Administration Period

The capsules filled up with the test substance were orally administered to the test animals once daily via an oral gavage between 9:00-12:30. The test animals were fasted for 2-3 hrs prior to each administration. The administration period was 11 days.

(5) Preparation of the Test Samples

Approximately 10 mL of blood was collected from the cephalic vein of each dog on—14 and—7 day (the 1st and the 2nd week prior to administration) as well as 4, 8, and 12 days after administration. The animals were fasted for approximately 18 hrs before collection of the blood.

The collected blood was placed in a test tube and allowed to stand at room temperature for 30 min to 1 hour. Then the blood was centrifuged (approximately 1,600 g, for 10 min) and the resultant serum obtained was used for assays.

(6) Test Procedure

Nitric oxide synthesized by nitric oxide synthase (NOS) is rapidly converted to nitrate ion (NO₃⁻) and nitrite ion (NO₂⁻). Blood concentration of total nitric oxides (NOₓ) is represented by the sum of NO₃⁻ and NO₂⁻ assayed by HPLC.

In addition, total cholesterol content was assayed by enzymatic assay method while HDL, LDL, and triglyceride were determined by homogeneous method, chemically modified method, and enzymatic techniques, respectively. ALP was assayed by Bessey-Lowry method. Clinical Laboratory System (TBA-120FR, Toshiba Medical Systems Corporation) was used in all of these determinations.

(Results)

Relative values of blood concentration of total nitric oxides (NOₓ) in animals treated with each dose of simvastatin or atorvastatin calcium, gamma-oryzanol, and benfotiamine alone as well as their medicinal compositions as described above were calculated against each converted average value calculated from that determined 2 weeks and one week before treatment into 100.

In addition, relative values of blood concentrations of various lipids in animals treated with each dose of simvastatin or atorvastatin calcium, gamma-oryzanol, and benfotiamine alone as well as their medicinal compositions as described above were calculated against each converted average value calculated from that determined 2 weeks and one week before treatment into 100.

The results are summarized in Tables 1-5. The values indicate average results calculated from 5 dogs per group.

TABLE 1

| | Changes in blood NOₓ levels (%) | | |
|---|---|---|---|
| | | | 12 Days |
| | 4 Days after | 8 Days after | after |
| Test substance (mg/Kg) | treatment | treatment | treatment |
| Gamma-oryzanol (100) | 105.3 | 111.7 | 102.4 |
| Simvastatin (1) | 101.6 | 85.5 | 121.9 |
| Simvastatin (1) + Gamma- | 135.2 | 131.9 | 141.4 |
| oryzanol (100) | | | |

TABLE 2

| | Changes in blood NOₓ levels (%) | | |
|---|---|---|---|
| | | 8 Days | 12 Days |
| | 4 Days after | after | after |
| Test substance (mg/Kg) | treatment | treatment | treatment |
| Benfotiamine (50) | 96.4 | 91.6 | 107.1 |
| Simvastatin (1) | 101.6 | 85.5 | 121.9 |
| Simvastatin (1) + Benfotiamine | 107.7 | 136.1 | 126.6 |
| (50) | | | |
| Atorvastatin calcium (2) | 117.7 | 114.1 | 117.9 |
| Atorvastatin calcium (2) + | 101.2 | 118.0 | 162.3 |
| Benfotiamine (50) | | | |

TABLE 3

| | Changes in blood total cholesterol levels (%) | | |
|---|---|---|---|
| | 4 Days after | 8 Days after | 12 Days after |
| Test substance (mg/Kg) | treatment | treatment | treatment |
| Gamma-oryzanol (100) | 98.8 | 98.5 | 101.9 |
| Simvastatin (1) | 94.8 | 94.1 | 92.4 |
| Simvastatin (1) + Gamma- | 94.3 | 87.1 | 86.8 |
| oryzanol (100) | | | |

TABLE 4

| | Changes in blood LDL levels (%) | | |
|---|---|---|---|
| | 4 Days after | 8 Days after | 12 Days after |
| Test substance (mg/Kg) | treatment | treatment | treatment |
| Gamma-oryzanol (100) | 100.4 | 98.5 | 98.3 |
| Simvastatin (1) | 83.9 | 90.4 | 81.3 |
| Simvastatin (1) + Gamma- | 84.7 | 69.2 | 69.0 |
| oryzanol (100) | | | |

TABLE 5

| | Changes in atherosclerosis index | | |
|---|---|---|---|
| | (LDL/HDL) (%) | | |
| | 4 Days after | 8 Days after | 12 Days after |
| Test substance (mg/Kg) | treatment | treatment | treatment |
| Gamma-oryzanol (100) | 101.4 | 99.5 | 95.9 |
| Simvastatin (1) | 86.6 | 93.8 | 86.3 |
| Simvastatin (1) + Gamma- | 87.4 | 76.1 | 75.8 |
| oryzanol (100) | | | |

As shown clearly in Table 1 and Table 2, the production of vascular endothelium nitric oxide was promoted and/or blood concentration of vascular endothelial nitric oxide was maintained or increased following combined administration of simvastatin or atorvastatin with gamma-oryzanol or benfotiamine.

In addition, as clearly shown in Table 3-Table 5, blood lipid levels were lowered following combined administration of simvastatin with gamma-oryzanol.

[Industrial Applicability]

Since the medicinal compositions containing an HMG-CoA reductase inhibitor and gamma-oryzanol and/or thiamine derivative of the present invention exert potent promoting effects on vascular endothelial nitric oxide synthesis, maintain or increase blood concentration of vascular endothelial nitric oxide, and mitigate blood lipid levels, the medicinal compositions of the present invention are useful as preventive or therapeutic agents against diseases caused by decreases in vascular endothelial nitric oxide synthase activity and/or decreases in concentration of vascular endothelial nitric oxide, diseases wherein decreases in vascular endothelial nitric oxide synthase activity, and/or decreases in concentration of vascular endothelial nitric oxide are elicited, or diseases caused by high blood lipids. For example, the medicinal compositions comprising an HMG-CoA reductase inhibitor and gamma-oryzanol and/or thiamine derivative of the present invention are useful for the prevention or treatment of symptoms of diseases, wherein nitric oxide production was decreased by cardiovascular diseases such as hypertension, hypercholesterolemia, atherosclerosis, ischemic heart disease, cardiac failure, thrombosis, pulmonary hypertension, restenosis, peripheral circulatory disorder, and the like; cerebrovascular diseases such as pulmonary hypertension cerebral ischemia, cerebral infarction, cerebrovascular disorders, senile dementia, and the like; disorders of the kidney and urinary tract such as renal dysfunction, impotency, and the like; diabetes mellitus; or certain drugs.

1. A pharmaceutical composition comprising effective amounts of an HMG-CoA reductase inhibitor; and gamma-oryzanol and/or a thiamine derivative.

2. The pharmaceutical composition according to claim 1 in which the HMG-CoA reductase inhibitor is one or more HMG-CoA reductase inhibitors selected from the group consisting of pravastatin, lovastatin, simvastatin, fluvastatin, rivastatin, atorvastatin, pitavastatin, and rosuvastatin.

3. The pharmaceutical composition according to claim 1 in which the HMG-CoA reductase inhibitor is atorvastatin.

4. The pharmaceutical composition according to claim 1, in which the HMG-CoA reductase inhibitor is simvastatin.

5. The pharmaceutical composition according to claim 2, in which the thiamine compound is one or more selected from the group consisting of thiamine, dicethiamine, octotiamine, cycotiamine, bisibuthiamine, bisbenthiamine, fursultiamine, prosultiamine, benfotiamine, and pharmacologically acceptable salts thereof.

6. The pharmaceutical composition according to claim 5, in which the thiamine compound is benfotiamine.

7. The pharmaceutical composition according to claim 5, in which an HMG-CoA reductase inhibitor and gamma-oryzanol are present as essential active ingredients.

8. The pharmaceutical composition of claim 1 in solid dosage form containing 0.005-3% HMG-CoA reductase inhibitor; and one selected from the group consisting of 0.5-90% gamma-oryzanol and 0.2-40% thiamine compound.

9. The pharmaceutical composition of claim 1 in liquid or solution dosage form containing 0.005-5 mg/mL; and one selected from the group consisting of 2-200 mg/mL gamma-oryzanol and 1-100 mg/mL thiamine compound.

10. A method for promoting production of vascular endothelial nitric oxide and/or maintaining or increasing blood concentration of vascular endothelial nitric oxide and/or mitigating blood lipid levels, comprising administering an effective amount of an HMG-CoA reductase inhibitor; and gamma-oryzanol and/or a thiamine compound simultaneously or separately at a certain time interval, to a mammal in need thereof.

11. The method according to claim 10, wherein blood lipids levels are mitigated.

12. The method according to claim 10, wherein diseases caused by decreases in vascular endothelial nitric oxide synthase activity and/or decreases in blood concentration of vascular endothelial nitric oxide are prevented or treated.

13. The method according to claim 10, wherein diseases that decrease vascular endothelial nitric oxide synthase activity and/or blood concentration of vascular endothelial nitric oxide, are prevented or treated.

14. The method according to claim 10, wherein decreases of nitric oxide production caused by cardiovascular diseases, cerebrovascular diseases, disorders of the kidney and urinary tract, diabetes mellitus, or certain drugs, are prevented or treated.

15. The method according to claim 10, wherein diseases caused by high blood lipid levels are prevented or treated.

16. The method according to claim 10, wherein hypercholesterolemia or atherosclerosis are prevented or treated.

17. The method according to claim 10, comprising administering to an adult human in need thereof, in one or more doses, a total of 1-200 mg/day HMG-CoA reductase inhibitor; and either 10 1000 mg/day gamma-oryzanol or 0.5-500 mg/day of a thiamine compound selected from the group consisting of thiamine, dicethiamine, octotiamine, cycotiamine, bisibuthiamine, bisbenthiamine, fursultiamine, prosultiamine, benfotiamine and pharmacologically acceptable salts thereof.
